(19) United States
(12) Patent Application Publication
(10) Pub. No.: US 2009/0035235 A1
(43) Pub. Date: Feb. 5, 2009

(54) Cosmetic composition for combating the cutaneous consequences of pollution

(76) Inventor: Olivier Courtin, Neuilly-Sur-Seine (FR)

Correspondence Address: HARNESS, DICKEY & PIERCE, P.L.C., P.O. BOX 828, BLOOMFIELD HILLS, MI, 48303, US

(21) Appl. No.: 11/918,860

(22) PCT Filed: Apr. 21, 2006
(86) PCT No.: PCT/FR2006/000896

§ 371 Date: Oct. 19, 2007

(30) Foreign Application Priority Data
Apr. 22, 2005 (FR) 0551037

(51) Int. Cl. A61K 8/97; A61Q 17/00; A61Q 17/04

(52) U.S. Cl. 424/59; 424/729

(57) ABSTRACT

The invention relates to the use of the extracts of plants of the genus Lapsana and of the species Lapsana communis L (Asteraceae) in particular. Said extracts are preferably obtained by maceration in a hydroglycolic mixture, and are used in cosmetic or dermopharmaceutical preparations for their skin-protective and radical-resistant properties. The actions of such preparations produce anti-ageing and anti-wrinkle effects, and protect the basic constituents of the cutaneous structure from the harsh effects of oxygen free radicals while prolonging the skin's suppleness and protective function. Said compositions supply protective cellular action against the deleterious effects of free radicals and those of enzymes such as elastase and hyaluronidase.

### CROSS REFERENCE TO RELATED APPLICATIONS

This application is a National Phase Entry of PCT/FR2006/000896, filed Apr. 21, 2006, which claims priority to French Application No. 05/51037, filed Apr. 22, 2005. Both of these application are incorporated by reference herein.

### BACKGROUND AND SUMMARY

The present invention relates to a new cosmetic composition that is capable of combating and preventing the effects of atmospheric pollution on the skin containing two vegetable extracts: Camellia sinensis and Lapsana communis.

The skin, with a surface area of around 2 m², is the largest organ of the human body. Essential for life, true interface between the body and its surroundings, it is an interactive barrier. It has great capacity for adaptation and for reacting towards aggressions. It is also a prime site for absorbing pollutants from the environment, passing substances that can have a direct toxic effect on the skin.

Pure air is a mix of 78% nitrogen, 21% oxygen, 0.09% argon, 0.1% water vapour and other more or less rare gases, including carbon dioxide, hydrogen and ozone. Atmospheric pollution is defined as a modification of pure air, through a modification of its components or through the addition of toxic elements. In a very simplistic manner, it can be said that the currently identified pollutants are essentially produced by industry, heating and the circulation of automobiles. They are usually classified into seven chemical families:
Oxidising agents such as ozone or nitrogen oxides are irritants and generators of free radicals.
Dust consists of particles in suspension which sometimes join polycyclic hydrocarbons. Their harmful role is modified by the temperature and relative humidity of the air; the acid particles are irritant; a reduction in the moisturisation and oxygenation of the tissues can be observed.
Organic chemical products are carcinogens; they come from industrial waste and the combustion process in automobiles.
Carbon monoxide produces tissue hypoxia; 80% of it comes from automobiles.
Hydrocarbons and solvents represent 50% of the pollution from automobiles; they are toxic, irritant, carcinogenic and mutagenic. In the presence of light, they react with nitrogen oxides to produce ozone.
Sulphur dioxide is one of the products of the combustion of oil and coal; it causes an alteration of the hydrolipidic film and irritations.
Metals such as lead, zinc, aluminium, mercury, . . . can interfere with cell metabolism, by attacking enzymatic reactions. They contribute to oxidative damage with lesions to DNA and cellular lipids.

For all these pollutants, the risk varies according to their concentration, the duration of exposure, their associations and, especially, the predisposition of the subject. They have synergy of action among each other.

The effects of pollution on the skin are varied: one can observe acidification of the skin pH, reduced moisturisation with increased transepidermal loss of water, increased desquamation, reduced flexibility of the stratum corneum, a modification of surface lipids due to the action of free radicals, a reduction of the cellular energy metabolism. Repetition of these aggressions begins an inflammatory process and predisposes to reactions of intolerance. Thus, a cosmetic composition that is capable of combating and preventing the effects of atmospheric pollution on the skin must be able to combat these various phenomena. The work conducted by the applicant has revealed that the association of two vegetable extracts of Camellia sinensis and Lapsana communis has an effect that protects against the effects of pollution on the skin.

The invention therefore relates, most particularly, to a composition containing an extract of Camellia sinensis and Lapsana communis. Camellia sinensis, also known as white tea, is native to Southeast Asia and the variety used in the composition according to the invention is Pai Mu Tan. It belongs to the family Theaceae, which also includes green tea and black tea. It is different from these other types in the way it is prepared, limited to minimalist operations that ensure it conserves its high content of polyphenols. The extract of Camellia sinensis used in the composition according to the invention is obtained by hydroglycolic extraction of the leaves of white tea. This produces a translucent, dark orange solution with a characteristic smell. This solution is titrated in total polyphenols and in PCO (proanthocyanidin oligomers); it has the following analytic characteristics:
dry matter: 2 to 4%
pH: 5.3 to 7.3 proportion of total polyphenols: >5 g/1 gallic acid equivalent
proportion of PCOs: 2.5 g/1 catechin equivalent.

Lapsana communis, of the family Asteraceae, is a very common annual plant, growing at roadsides and other locations subjected to atmospheric pollution. It is an undemanding plant and is very hardy. The extract of Lapsana communis used in the composition according to the invention is obtained by hydroglycolic extraction of the aerial part of the plant. In this way, a clear brown liquid with a characteristic odour is obtained, having the following analytic characteristics:
water content: 18 to 26%
pH: 6 to 8
density: 1.055 to 1.075
refractive index: 1.420 to 1.440

The composition according to the invention contains:
of the order of 0.01 to 5% by weight, preferably 0.01 to 0.5% by weight and most preferably 0.01 to 0.05% by weight of Camellia sinensis extract and
of the order of 0.01 to 5% by weight, preferably 0.01 to 0.5% by weight and most preferably 0.01 to 0.05% by weight of Lapsana communis extract.

The compositions according to the invention can also comprise one or more formulation agents or additives commonly and traditionally used in cosmetic and dermatological compositions such as, as a non-limiting example, demulcents, colorants, film-forming agents, surface-active agents, perfumes, preservatives, emulsifiers, oils, glycols, vitamins such as vitamin E, UV filters, etc. Using their knowledge in the field of cosmetics, those skilled in the art will know what formulation agents to add to the compositions of the invention and in what quantities according to the desired properties.

The compositions according to the invention can be presented in any form known to those skilled in the trade in the field of cosmetology and dermatology, with the only galenic restriction being application on facial or body skin. Advantageously, the compositions according to the invention are presented in the form of a gel, lotion, cream, emulsion, milk, spray, etc.

The present invention also relates to the cosmetic use of a composition according to the invention to combat and prevent the effects of pollution on the skin. The present invention also relates to the use of extracts of Camellia sinensis and Lapsana communis for the preparation of a cosmetic composition for combating and preventing the effects of pollution on the skin. Finally, the present invention relates to a method of cosmetic care, for combating and preventing the effects of pollution on the skin, characterised in that an adequate amount of a composition according to the invention is applied to the skin.

The following examples are provided by way of example and cannot be interpreted as limiting the scope of the invention. They relate, on the one hand, to an assessment of the antiradical effect of the Camellia sinensis and Lapsana communis complex on human keratinocytes in culture and an assessment of the effect of this same complex on the energetic metabolism and, on the other hand, to examples of compositions included in the present invention.

### BRIEF DESCRIPTION OF THE DRAWINGS

The examples are given in reference to the following figures, wherein:

FIG. 1 shows the effects of the Camellia sinensis and Lapsana communis complex (CLALAMP complex) on the reversibility of the cytotoxic effect of exhaust gases on keratinocytes in culture. The Camellia sinensis and Lapsana communis complex was tested at 0.01%, 0.025% and 0.05%;

FIG. 2 shows the antiradical effects of the Camellia sinensis and Lapsana communis complex (CLALAMP complex tested at 0.01%, 0.025% and 0.05%) by measurement of malondialdehyde (MDA), a lipoperoxidation marker, in keratinocytes in culture previously exposed to exhaust gases;

FIG. 3 shows the effect of the Camellia sinensis and Lapsana communis complex (CLALAMP complex tested at 0.01%, 0.025% and 0.05%) on the respiration rate of keratinocytes in culture previously exposed to exhaust gases. The results are given in picoatoms of oxygen per million cells and per minute;

FIG. 4 shows the effect of the Camellia sinensis and Lapsana communis complex (CLALAMP complex tested at 0.01%, 0.025% and 0.05%) on the mitochondrial respiration rate in the presence of pyruvate-malate of keratinocytes in culture previously exposed to exhaust gases. The results are given in picoatoms of oxygen per million cells and per minute;

FIG. 5 shows the effect of the Camellia sinensis and Lapsana communis complex (CLALAMP complex tested at 0.01%, 0.025% and 0.05%) on the cellular basal ATP synthesis of keratinocytes in culture previously exposed to exhaust gases. The results are given in nmoles per million cells and per minute; and

FIG. 6 shows the effect of the Camellia sinensis and Lapsana communis complex (CLALAMP complex tested at 0.01%, 0.025% and 0.05%) on the cellular mitochondrial ATP synthesis rate of keratinocytes in culture previously exposed to exhaust gases. The results are given in nmoles per million cells and per minute.

### DETAILED DESCRIPTION

I. Assessment of the Antiradical Effect of the Camellia sinensis and Lapsana communis Complex on Human Keratinocytes in Culture.

A. Material and methods.

Material.

The keratinocyte cultures are obtained from human foreskin cells collected during circumcision and amplified in a KGM2 medium (Clonetics) supplemented with insulin, EGF and pituitary extract. The assays were conducted on keratinocytes between the 2^{nd} and 4^{th} passages in order to guarantee the reproducibility of the various experiments. The exhaust gases were produced by a motor. The gases were placed in contact with human keratinocytes in culture for 2 hours. The cells were then incubated with or without the product being studied for a further 22 hours.

Cytotoxicity Study.

This step was conducted using the Formazan blue assay (MTT). After 24 hours of cell incubation in the presence or absence of the product being studied at different concentrations, the wells containing the cells were emptied by slowly turning them over and the cell layer was then rinsed with the culture medium. 200 μl of a diluted MTT solution were distributed in all the wells. The plates were then incubated at 37° C. for 2 to 4 hours. The formation of Formazan blue crystals could then be observed, in a quantity in inverse proportion to the succinate dehydrogenases obtained. The wells where then emptied again by slowly turning them over; the cells were then lysed and the Formazan blue crystals dissolved, by adding 200 Al of dimethyl sulfoxide (DMSO). After homogenising the colour, by agitation, the plates were observed at 570 nm using a spectrophotometer.

The assay was conducted after 24 hours of contact between the Camellia sinensis and Lapsana communis complex and the cells.

Batch 1: negative control not receiving any product

Batch 2: cells exposed to the exhaust gases

Batch 3: cells exposed to the exhaust gases and then treated with the complex (0.01%)

Batch 4: cells exposed to the exhaust gases and then treated with the complex (0.025%)

Batch 5: cells exposed to the exhaust gases and then treated with the complex (0.05%)

Study of the Antiradical Activity.

The assay was conducted in triplicate after 24 hours of contact between the Camellia sinensis and Lapsana communis complex and the cells.

Description of the Batches:

Batch 1: negative control not receiving any product

Batch 2: cells exposed to the exhaust gases

Batch 3: cells exposed to the exhaust gases and then treated with the complex (0.01%)

Batch 4: cells exposed to the exhaust gases and then treated with the complex (0.025%)

Batch 5: cells exposed to the exhaust gases and then treated with the complex (0.05%)

a) Extraction of Malondialdehyde (MDA).

After 24 h of contact between the product and the cells, the latter were returned to suspension in:
250 μl of Tris buffer, 50 mM, pH 8 containing NaCl 0.1 M; EDTA 20 mM
25 μl of SDS at 7%
300 μl of HCl (0.1 N)
38 μl of phosphotungstic acid at 1% in water
300 μl of thiobarbituric acid at 0.67% in water

After 1 hour of incubation in the dark at 50° C. and cooling in ice-cold water, 300 ml of n-butanol was added to each tube. These were centrifuged at 10,000 g and 0° C. for 10 min. The top phase was recovered for titrating the MDA.

b) Titrating the Malondialdehyde (MDA).

The MDA was titrated by measuring the fluorescence after separating the MDA-TBA complex by HPLC.
Bischoff Pump Model 2.200
Automatic alcohol injector Model 788 autosampler
Ultrasep C18 (30 cm×0.18 cm) column, 6 mm of porosity
Fluorescence detector, Jasco 821-F1

The fluorescence detection was conducted with excitation at 515 nm and emission at 553 nm. The eluent used consisted of methanol:water, 40:60 (v/v) with the pH adjusted using KOH 1 M. Quantification was performed using standards treated as the samples (0.125, 0.25, 0.5 and 1 mM) using an ICS software application (Pic 3) (Instrumentation, Consommable Service).

B. Results.

1. Cytotoxicity Study.

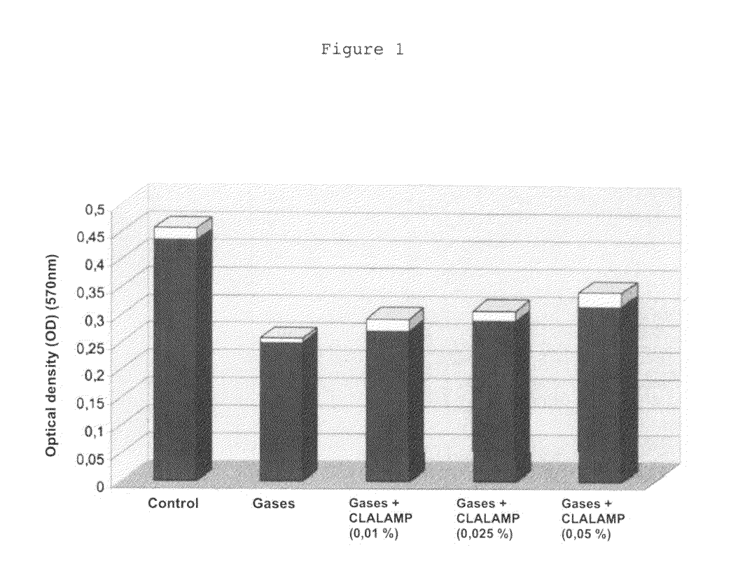

The aim of this step was to discover the reversibility of the cytotoxic effect of the exhaust gases after treatment with the Camellia sinensis and Lapsana communis complex on human keratinocytes in culture. This study was conducted by determining cellular viability using the MTT assay. The Camellia sinensis and Lapsana communis complex incubated at respective concentrations of 0.025 and 0.05% with the keratinocytes had a significant effect on the reversibility of the cytotoxicity induced by the exhaust gases. This inhibition of cytotoxicity results in cellular viability increases of 16 and 27% respectively (FIG. 1).

2. Study of the Antiradical Activity.

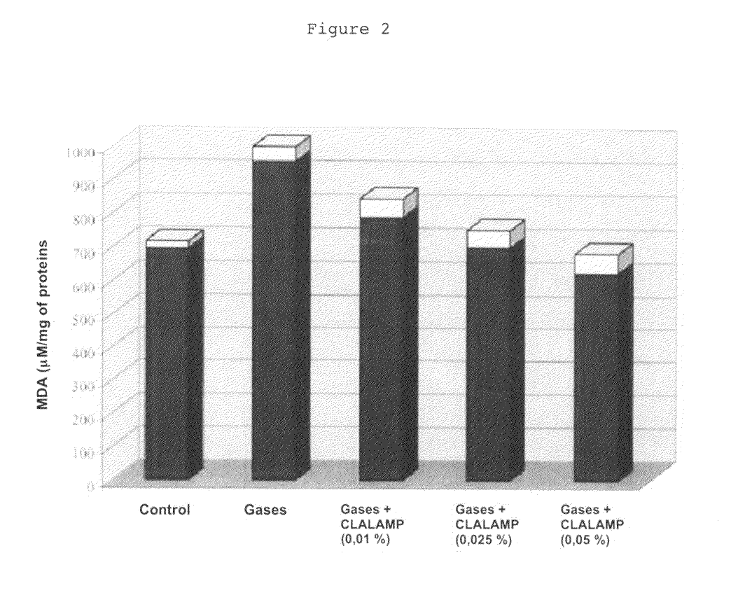

The results obtained show that the Camellia sinensis and Lapsana communis complex (tested respectively at concentrations of 0.01, 0.025 and 0.05%) provides considerable protection against the lipoperoxidation caused by the exhaust gases. The MDA production reduction percentage is −17, −27 and −34% respectively (FIG. 2).

3. CONCLUSIONS.

In the selected experimental conditions, the Camellia sinensis and Lapsana communis complex appears to have an effect on reversing the cytotoxicity and pro-radical activity induced by the exhaust gases on the human keratinocytes in culture after 24 hours of contact. Indeed, the MDA titrations show that the Camellia sinensis and Lapsana communis complex studied at concentrations of 0.01, 0.025 and 0.05% has induced considerable protection for the human keratinocytes against the lipoperoxidation caused by the exhaust gases. The MDA production reduction percentage is −17, −27 and −34%. In conclusion, the Camellia sinensis and Lapsana communis complex has a considerable antiradical effect on the free radicals induced by the exhaust gases.

II. Assessment of the Effect of the Camellia sinensis and Lapsana communis Complex on the Energetic Metabolism.

A. Material and Methods.

1. Material.

The keratinocyte cultures are obtained from human foreskin cells collected during circumcision and amplified in a KGM2 medium (Clonetics) supplemented with insulin, EGF and pituitary extract. The assays were conducted on keratinocytes between the 2^{nd} and 4^{th} passages in order to guarantee the reproducibility of the various experiments. The exhaust gases were produced by a motor. The cells were placed in contact with the gases for 2 hours. They were then incubated with or without the product being studied for a further 22 hours.

2. Study of the effect of the Camellia sinensis and Lapsana communis Complex on the Respiration Rate (Consumption of Oxygen in Picoatoms of Per Million Cells and Per Minute).

This assay was conducted under 2 different conditions:
effect on the basal cellular respiration rate in non-permeabilised cells in the presence of glucose,
effect on the mitochondrial respiration rate of permeabilised cells in the presence of the pyruvate-malate respiration substrate.

This assay was conducted on keratinocytes in culture dissociated in trypsin. 5 to 10 million keratinocytes in culture were placed in suspension in 1 ml of HBSS medium at 30° C. containing glucose (basal respiration) or pyruvate-malate (mitochondrial respiration). Respiration was monitored in real time and given in picoatoms of oxygen consumed per minute and per 10⁶ cells. Adding different quantities of the product to the tank of the oxygraph shows possible stimulation or inhibition of respiration.

The amount of oxygen dissolved in an incubation medium was determined using a Clark electrode. The oxygen diffused through a Teflon film is reduced at the polarised platinum cathode at −0.8 volts. Under these conditions, the current passing between this cathode and the silver anode is proportional to the oxygen concentration in the solution. The ion bridge is provided by a semi-saturated solution of KCl. The measurements are taken and processed by a microcomputer.

Description of the Batches:

Batch 1: negative control not receiving any product

Batch 2: cells exposed to the exhaust gases

Batch 3: cells exposed to the exhaust gases and then treated with the complex (0.01%)

Batch 4: cells exposed to the exhaust gases and then treated with the complex (0.025%)

Batch 5: cells exposed to the exhaust gases and then treated with the complex (0.05%)

The assay was conducted after 20 minutes of contact between the product—the Camellia sinensis and Lapsana communis complex—and the cells.

3. Study of the Effect of the Product on the Basal and Mitochondrial ATP Synthesis of the Keratinocytes in Culture.

The aim of this assay is to assess the effect of the Camellia sinensis and Lapsana communis complex on the basal and mitochondrial ATP synthesis rate of keratinocytes in culture. This is determined by means of bioluminescence using the luciferin/luciferase kit. The amount of newly synthesised and basal ATP in the various aliquots is measured by the light emitted during the following ATP consumption reaction:

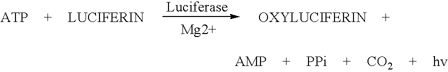

The intensity of the light emitted during this reaction is measured using a luminometer with the ATP monitoring reagent (ATP Bioluminescence Assay Kit HS II) from Boehringer Mannheim. This device transcribes the light emitted during the reaction into RLUs (relative luminosity units). The measured RLUs are converted into moles of ATP according to a standard ATP scale. The ATP synthesis rate is given in nmoles/min/10⁶ cells. Keratinocytes in culture were cultivated in a CO₂ incubator at the rate of 10⁶ per run in an ADM culture medium (Clonetics).

The treatment consists of directly applying the Camellia sinensis and Lapsana communis complex at the desired concentration to the cells in suspension in the tank of the oxygraph. The cells at a concentration of 10⁶ cells/ml are placed in suspension in a “respiration buffer” (Hanks-Hepes glucose 20 mM), in the tank of the oxygraph with the thermostat set to 30° C. and agitated. The cells are permeabilised using digitonin. The addition of a respiration substrate (pyruvate 10 mM and malate 10 mM) allows the oxygen consumption rate to be observed (state 2 according to Chance). After adding different quantities of the product (final concentrations: 0.01, 0.025 and 0.05%) to the tank of the oxygraph at regular intervals, one aliquot is taken from the tank of the oxygraph to titrate its ATP according to the method described above. The addition of different quantities of the product to the tank of the oxygraph therefore makes it possible to show the possible activation or inhibition of ATP synthesis.

Description of the Batches:

Batch 1: negative control not receiving any product

Batch 2: cells exposed to the exhaust gases

Batch 3: cells exposed to the exhaust gases and then treated with the complex (0.01%)

Batch 4: cells exposed to the exhaust gases and then treated with the complex (0.025%)

Batch 5: cells exposed to the exhaust gases and then treated with the complex (0.05%)

B. Results.

1. Study of the Effect of the Complex at Different Doses on the Respiration Rate (Oxygen Consumption) of Keratinocytes in Culture.

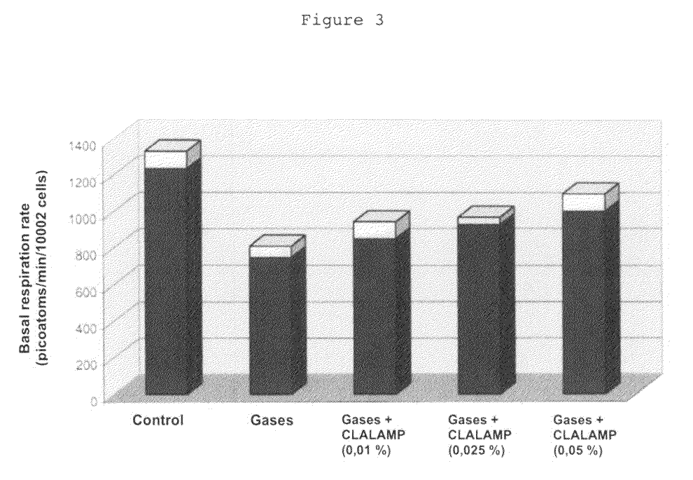

This assay was conducted under 2 different experimental conditions. The results are given in picoatoms of oxygen per million cells and per minute. The Camellia sinensis and Lapsana communis complex, incubated respectively at concentrations of 0.01, 0.025 and 0.05% with the keratinocytes previously exposed to exhaust gases, has induced a considerable effect on the oxygen consumption rate at the studied concentrations. This stimulation of basal respiration produces a substantial increase of the oxygen consumption rate: 14, 24 and 34% respectively (FIG. 3).

2. Effect of the Complex on the Mitochondrial Respiration Rate.

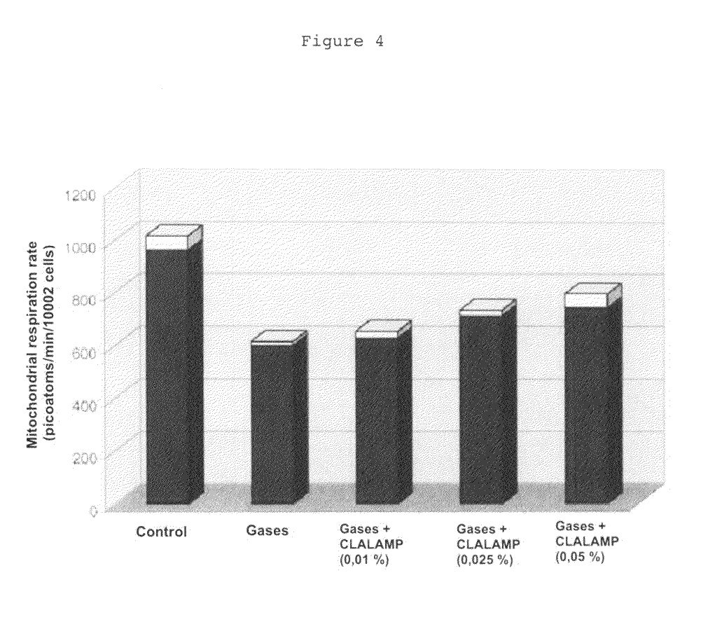

This assay was conducted on the permeabilised cells in the presence of the pyruvate-malate respiration substrate. The Camellia sinensis and Lapsana communis complex, incubated at various concentrations with the keratinocytes previously exposed to exhaust gases, has induced a considerable effect on the oxygen consumption rate at the concentrations of 0.025 and 0.05%. This stimulation of mitochondrial respiration produces a substantial increase of the oxygen consumption rate: 19 and 24% respectively (FIG. 4).

3. Effect of the Complex at Different Doses on Basal and Mitochondrial ATP Synthesis of the Keratinocytes in Culture.

This assay was conducted under 2 different experimental conditions. The results are given in nmoles per minute and per million cells.

a) Effect on Cellular Basal A TP Synthesis.

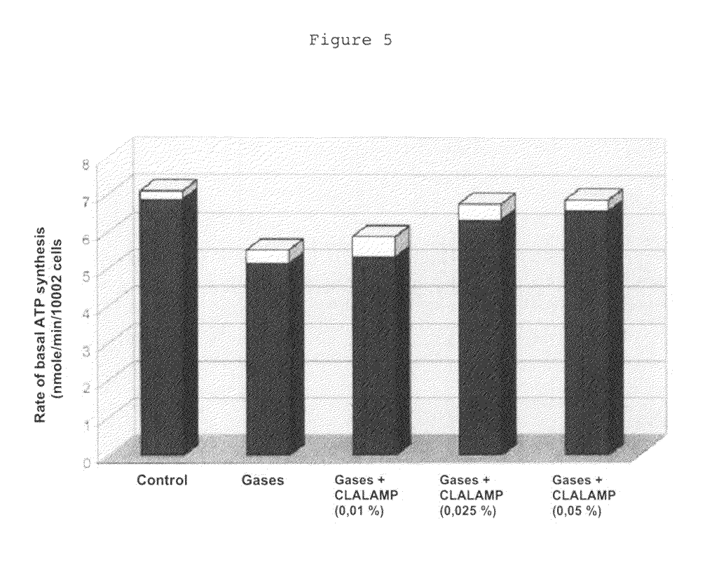

This assay was conducted on non-permeabilised whole cells in the presence of glucose. The Camellia sinensis and Lapsana communis complex, incubated at various concentrations with the keratinocytes previously exposed to exhaust gases, has induced a considerable effect on the basal ATP synthesis rate at the concentrations of 0.025 and 0.05%. The basal ATP synthesis rate increased by 23 and 29% respectively (FIG. 5).

b) Effect on the Rate of Cellular Mitochondrial ATP Synthesis.

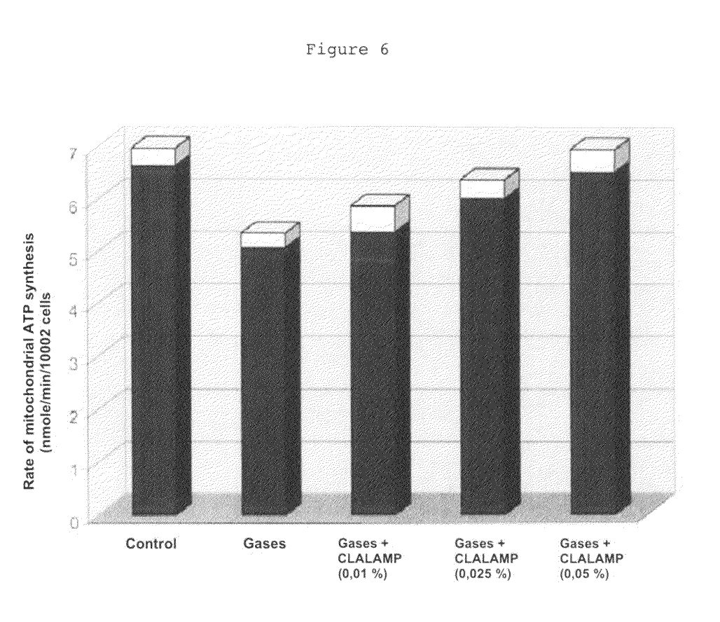

This assay was conducted on permeabilised whole cells in the presence of pyruvate-malate. The Camellia sinensis and Lapsana communis complex, incubated at various concentrations with the keratinocytes previously exposed to exhaust gases, has induced a considerable effect on the mitochondrial ATP synthesis rate at the concentrations of 0.025 and 0.05%. The mitochondrial ATP synthesis rate increased by 19 and 29% respectively (FIG. 6).

III. Conclusions Regarding the Results Obtained.

Under the experimental conditions, in view of the results. obtained, the Camellia sinensis and Lapsana communis complex has induced the following:
a considerable effect on the reversibility of the cytotoxicity induced by the exhaust gases, which is 16 and 27% respectively for concentrations of the Camellia sinensis and Lapsana communis complex of 0.025 and 0.05% (FIG. 1),
a considerable effect on the basal respiration rate, which is 24 and 34% respectively for concentrations of the Camellia sinensis and Lapsana communis complex of 0.025 and 0.05% (FIG. 3),
a considerable effect on the mitochondrial respiration rate, which is 19 and 24% respectively for concentrations of the Camellia sinensis and Lapsana communis complex of 0.025 and 0.05% (FIG. 4),
a considerable effect on the basal ATP synthesis rate, which is 23 and 29% respectively for concentrations of the Camellia sinensis and Lapsana communis complex of 0.025 and 0.05% (FIG. 5),
a considerable effect on the mitochondrial ATP synthesis rate, which is 19 and 29% respectively for concentrations of the Camellia sinensis and Lapsana communis complex of 0.025 and 0.05% (FIG. 6).

It is noted that, surprisingly, the Camellia sinensis and Lapsana communis complex is active at very low concentrations, a sign of the synergy between the two extracts.

IV. Examples of Compositions According to the Invention.

A. Anti-Pollution Gel Cream.

| DEMINERALISED WATER | qs 100 |
|---|---|
| PEMULEN TR1 | 0.5 |
| GLYCERINE | 5.0 |
| SEPIGEL 305 | 1.0 |
| EXTRACT OF Camellia sinensis | 0.05 |
| EXTRACT OF Lapsana communis | 0.05 |
| ISONONYL ISONONANOATE | 7.0 |
| C12-C15 ALKYL BENZOATE | 3.0 |
| SILICONE OIL | 2.0 |
| SODIUM HYDROXIDE | 0.2 |
| PERFUME | 0.3 |
| COLORANTS AT 1% | 0.12 |
| PRESERVATIVES | 1.0 |

B. Anti-Pollution Cream.

| | |
|---|---|
| DEMINERALISED WATER | qs 100 |
| CETEARYL GLUCOSIDE | 5.0 |
| TRIGLYCERIDES C8-C10 | 10 |
| SILICONE OIL | 2.0 |
| VOLATILE SILICONE | 5.0 |
| CARBOMER | 0.2 |
| GLYCERINE | 5.0 |
| EXTRACT OF Camellia sinensis | 0.05 |
| EXTRACT OF Lapsana communis | 0.05 |
| PERFUME | 0.3 |
| PRESERVATIVES | 1.0 |

1-8. (canceled)

9. A cosmetic composition comprising an extract of Camellia sinensis and an extract of Lapsana communis.

10. The cosmetic composition according to claim 9, wherein the Camellia sinensis extract is a hydroglycolic extract of leaves of this plant.

11. The cosmetic composition according to claim 9, wherein the Lapsana communis extract is a hydroglycolic extract of an aerial part of this plant.

12. The cosmetic composition according to claim 9, further comprising:
of the order of 0.01 to 5% by weight, preferably 0.01 to 0.5% by weight and most preferably 0.01 to 0.05% by weight of the Camellia sinensis extract; and
of the order of 0.01 to 5% by weight, preferably 0.01 to 0.5% by weight and most preferably 0.01 to 0.05% by weight of the Lapsana communis extract.

13. The cosmetic composition according to claim 9, further comprising one or more formulation agents or additives such as demulcents, colorants, film-forming agents, surface-active agents, perfumes, preservatives, emulsifiers, oils, glycols, vitamins and UV filters.

14. The cosmetic composition according to claim 9, wherein the composition combats and substantially prevents the effects of pollution on the skin.

15. A cosmetic composition comprising a hydroglycolic extract of the leaves of Camellia sinensis and a hydroglycolic extract of an aerial part of Lapsana communis, wherein the composition assists in preventing the effects of pollution on a skin of a user.

16. The cosmetic composition according to claim 15, wherein the extract of Camellia sinensis is titrated in total polyphenols and in proanthocyanidin oligomers.

17. The cosmetic composition according to claim 15, wherein the extract of Camellia sinensis has a pH of about 5.3 to 7.3.

18. The cosmetic composition according to claim 15, wherein the extract of Camellia sinensis has a proportion of total polyphenols greater than 5 g/1 gallic acid equivalent.

19. The cosmetic composition according to claim 15, wherein the extract of Lapsana communis has water content of about 18 to 26%.

20. The cosmetic composition according to claim 15, wherein the extract of Lapsana communis has a pH of about 6 to 8%.

21. The cosmetic composition according to claim 15, wherein the extract of Lapsana communis has a refractive index of about 1.420 to 1.440.

22. The cosmetic composition according to claim 15, further comprising:
of the order of 0.01 to 0.5% by weight of the Camellia sinensis extract; and
of the order of 0.01 to 0.5% by weight of the Lapsana communis extract.

23. The cosmetic composition according to claim 15, further comprising one or more formulation agents or additives taken from the group of: demulcents, colorants, film-forming agents, surface-active agents, perfumes, preservatives, emulsifiers, oils, glycols, vitamins and UV filters.

24. The cosmetic composition according to claim 15, further comprising a cream including the extracts.

25. The cosmetic composition according to claim 15, further comprising demineralised water, glycerine, sepigel, isononyl isononanoate, alkyl benzoate and silicone oil.

26. The cosmetic composition according to claim 15, further comprising demineralised water, cetearyl glucoside, triglycerides, silicone oil, volatile silicone and glycerine.

27. A method of cosmetic care, for combating and substantially preventing the effects of pollution on the skin, further comprising applying an adequate amount of a composition according to skin, the composition comprising an extract of Camellia sinensis and an extract of Lapsana communis.

28. The method according to claim 27, further comprising using extracts of Camellia sinensis and Lapsana communis to prepare a cosmetic composition for combating and preventing the effects of pollution on the skin.

29. The method according to claim 27, further comprising combining the extracts with demineralised water, glycerine, sepigel, isononyl isononanoate, alkyl benzoate and silicone oil.

30. The method according to claim 27, further comprising combining the extracts with demineralised water, cetearyl glucoside, triglycerides, silicone oil, volatile silicone and glycerine.

31. The method according to claim 27, further comprising obtaining a hydroglycolic extract from leaves of a Camellia sinensis plant.

32. The method according to claim 27, further comprising obtaining a hydroglycolic extract from an aerial part of a Lapsana communis plant.
